(19) United States
(12) Patent Application Publication
(10) Pub. No.: US 2026/0318848 A1
(43) Pub. Date: Oct. 15, 2026

(54) METHOD FOR DETERMINING A SOLID FRACTION

(71) Applicant: Endress+Hauser Flowtec AG, Reinach (CH)

(72) Inventors: Elena Reinbacher, Freising (DE); Stefan Pflüger, München (DE); Philippe Kocher, Bartenheim (FR)

(21) Appl. No.: 19/485,028

(22) PCT Filed: May 3, 2024
(86) PCT No.: PCT/EP2024/062306

§ 371 Date: Nov. 17, 2025

(30) Foreign Application Priority Data
May 15, 2023 (DE) 10 2023 112 734.9

(51) Int. Cl. G01N 22/00; G01N 33/18; G06N 3/048; G06N 3/08

(52) U.S. Cl. CPC G01N 22/00; G01N 33/18; G06N 3/048; G06N 3/08

(57) ABSTRACT

The present disclosure relates to a computer-implemented method for determining a solid fraction in a flowable medium, of a microwave sensor, comprising: receiving an input data set having at least one effective permittivity value ε_{eff}, providing the input data set to a machine learning model, the machine learning model having an exchangeable, application-specific parameter set, the parameter set having at least one weighting factor wᵢ and at least one bias bᵢ; determining a solid fraction value δ of the solid in the medium using the machine learning model; generating an output data set comprising the solid fraction value δ. Furthermore, the invention relates to a machine learning model, a computer-implemented method for generating a training data set for a machine learning model and a microwave sensor.

The invention relates to a computer-implemented method for determining a solid fraction, a machine learning model for determining a solid fraction, a computer-implemented method for generating a training data set for a machine learning model for determining a solid fraction, a microwave sensor for determining a solid fraction, a computer program having instructions that, when the program is executed by a computer, cause the computer to carry out the method according to the invention, a computer-readable medium that contains instructions that, when executed by a computer, cause the computer to carry out the method according to the invention and a method for configuring a microwave sensor according to the invention.

It is possible, by means of microwaves, to ascertain the physical variables of permittivity and loss factor of a medium in a process line. From these two variables—measured either at one or over many different frequencies—it is possible to draw conclusions regarding application-specific parameters, for example the proportion of water in a mixture of water and other non-polar or weakly polar components or a solid content in a liquid medium.

The established transmission/reflection measurement is described in L. F. Chen, C. K. Ong, C. P. Neo, V. V. Varadan, V. K. Varadan-“Microwave Electronics, Measurement and Materials Characterization,” John Wiley & Sons Ltd., 2004. For this purpose, the microwave signal interfaces at two different positions at the medium in a container or measuring tube, the scatter parameters (transmission and optionally reflection) are measured between these interface structures, and the mentioned physical properties of the medium are calculated from the measured scatter parameters.

WO 2018 121927 A1 teaches a measuring assembly for analyzing properties of a flowing medium by means of microwaves. In addition to the microwave antennas, the measuring assembly has an electrically insulating lining layer on the inner peripheral surface of the measuring tube. This lining layer forms a dielectric waveguide via which at least part of the one microwave signal can travel from a first microwave antenna to a second microwave antenna. One application for such a measuring assembly is the determination of the proportions of solids in the liquid medium being conveyed. WO 2021/099152 A1 teaches a microwave antenna which has a front section in contact with the medium, via which the excitation signal is emitted into the medium.

DE102017131269A1 discloses a method for continuously determining the fat content of milk with varying solids contents using a Coriolis flowmeter and a microwave sensor. These types of measuring devices can be integrated into milk processing processes.

DE 42 27 727 A1 discloses a method for determining a state variable of a measuring liquid from its conductivity value σ and its temperature value T with the aid of a neural network of the type mentioned above for determining a state variable of a measuring liquid. With the aid of the known method, the measuring liquid is classified into surfactant-containing and surfactant-free. At the output of the neural network, either the value 1 (surfactant-containing measuring liquid) or the value 0 (surfactant-free measuring liquid) is present. For classifying the measuring liquid, the concentration of non-ionic surfactants in the measuring liquid is determined. In order to be able to determine the concentration of non-ionic surfactants, in addition to the conductivity and the temperature of the measuring liquid, the pH value and the turbidity of the measuring liquid are also supplied to the neural network via two additional inputs of the input layer. In the known method, therefore, no specific concentration values are determined and output via the output of the output layer with the aid of the neural network.

DE 198 35 137 C2 discloses a method for determining a state variable of a measuring liquid from its conductivity value σ and its temperature value T with the aid of a neural network having an input layer having at least two inputs for the conductivity value and the temperature value, having an output layer having an output for the state variable and having at least one hidden layer arranged between the input layer and the output layer and having a plurality of neurons connected between the inputs and the output, characterized in that the concentration of the ions dissolved in the measuring liquid is determined as the state variable, wherein, in each neuron, the input values of the inputs are in each case weighted with an input-side weighting factor, the weighted input values are summed and the sum value is passed through an activation function.

The invention is based on the object of also providing a solution for determining the solid fraction in a medium based on the cited prior art. The object is achieved by the method according to claim 1 and the objects of the independent claims.

The computer-implemented method according to the invention for determining a solid fraction in a flowable, in particular aqueous, medium, in particular by means of a microwave sensor, comprises:
Receiving an input data set having at least one effective permittivity value ε_{eff}, in particular ascertained from measurement data, of the medium, and a temperature value T of the medium;
Providing the input data set to a machine learning model,
   wherein the machine learning model comprises an exchangeable, application-specific parameter set,
   wherein the parameter set comprises at least one weighting factor wᵢ and at least one bias bᵢ;
Determining a solid fraction value δ of the solid in the medium by means of the machine learning model;
Generating an output data set comprising the solid fraction value δ;
Optional: Outputting the output data set, in particular the solid fraction value δ.

Advantageous embodiments of the invention are the subject matter of the dependent claims.

One embodiment provides that the input data set further comprises a frequency value f and/or an effective density value ρ_{eff} of the medium.

One embodiment provides that the parameter set results from a mathematical model applied in a learning phase of the machine learning model for describing the properties of heterogeneous mixtures consisting of an insoluble substance located in a carrier substance.

One embodiment provides that the mathematical model is selected from:
the Maxwell-Garnett model
the parallel Wiener equation
the serial Wiener equation

The machine learning model according to the invention for determining a solid fraction in a flowable, in particular aqueous, medium, in particular for use in the method according to the invention comprises:
an input layer having at least two inputs for an effective permittivity value ε of the medium, and a temperature value T of the medium;
an output layer having an output for a solid fraction value δ of the solid in the medium;
at least two hidden intermediate layers present between the input layer and the output layer, in each case having a number Nᵢ of connected neurons, wherein the machine learning model is configured to weight input values
   received in each neuron of a first intermediate layer of the at least two intermediate layers in each case with a weighting factor, to sum the weighted input values and a bias bᵢ to form a sum value, to convert the sum value into an output value through an activation function and to provide the output value to at least one neuron, in particular to all neurons, of a second intermediate layer of the at least two intermediate layers.
   wherein the weighting factors wᵢ and the bias b; form an exchangeable, application-specific parameter set.

One embodiment provides that a hyperbolic tangent function and/or a ReLU (rectified linear unit) function is used as the activation function.

One embodiment provides that the input layer comprises at least one further input for a frequency value f and/or an effective density value ρeff.

One embodiment provides that the machine learning model comprises exactly three intermediate layers,
wherein a total number of neurons of the three intermediate layers is selected from a range of 40 to 2000, in particular up to 1000, and preferably up to 200 neurons.

One embodiment provides that a first intermediate layer comprises a number of neurons selected from the range of 20 to 80,
wherein a second intermediate layer comprises a number of neurons selected from the range of 10 to 80,
wherein a third intermediate layer comprises a number of neurons selected from the range of 10 to 60,

One embodiment provides that the machine learning model comprises exactly four intermediate layers,
wherein a total number of the neurons of the four intermediate layers is selected from a range of 100 to 250 neurons.

One embodiment provides that a first intermediate layer comprises a number of neurons selected from the range of 20 to 60,
wherein a second intermediate layer comprises a number of neurons selected from the range of 50 to 80,
wherein a third intermediate layer comprises a number of neurons selected from the range of 20 to 60,
wherein a fourth intermediate layer comprises a number of neurons selected from the range of 10 to 50.

One embodiment provides that the machine learning model comprises a data size of less than or equal to 500 KB, with a deviation of less than 0.1% relative to the mathematical model serving as the basis for the learning phase.

The computer-implemented method according to the invention for generating a training data set for a machine learning model for determining a solid fraction δ of a solid in a flowable, in particular aqueous, medium, in particular for the machine learning model according to the invention, comprises:
Creating an input training data set,
   wherein the input training data set comprises an effective permittivity value ε_{eff} of the medium, a permittivity value ε_{f} of the solid, a permittivity value εₘ of the carrier medium, a solid fraction value δ and a temperature T,
   wherein at least the effective permittivity value ε_{eff} and the permittivity value ε_{f} are ascertained using a random number generator,
   wherein the solid fraction value δ is calculated by means of a mathematical model as a function of the effective permittivity value ε_{eff}, the permittivity value ε_{f} and the permittivity value εₘ.

One embodiment provides that the input training data set further comprises an additional parameter β, which is selected from a number range of −1 to 1. The additional parameter β specifies whether the mathematical model is the parallel or serial Wiener equation.

One embodiment provides that the mathematical model is selected from:
the Maxwell-Garnett model
the parallel Wiener equation
the serial Wiener equation

One embodiment provides that the input training data set further comprises an effective density ρ_{eff} of the medium.

One embodiment provides that the input training data set is created for temperatures from a temperature range of 1 to 100° C., in particular 10 to 60° C., and/or
wherein the effective permittivity value ε_{eff} and the permittivity value ε_{f} are selected so that a solid fraction value δ between 0 and 35% results.

The microwave sensor according to the invention for determining a solid fraction δ of a solid in a flowable, in particular aqueous, medium, having means for performing the method according to the invention comprises:
a transmitting antenna for generating a microwave signal passing through the medium;
a receiving antenna for receiving the microwave signal passing through the medium,
an evaluation electronics unit that is configured to ascertain an effective permittivity of the medium as a function of a provided propagation time and/or attenuation of the microwave signal,
   wherein the application-specific parameter set is stored interchangeably in the evaluation electronics unit.

The computer program according to the invention having instructions that, when the program is executed by a computer, cause the computer to carry out the computer-implemented method according to the invention for determining a solid fraction in a flowable, in particular aqueous, medium, in particular by means of a microwave sensor and/or the method according to the invention for generating a training data set for a machine learning model for determining a solid fraction δ of a solid in a flowable, in particular aqueous, medium, in particular for the machine learning model according to the invention.

The computer-readable medium according to the invention, which contains instructions that, when executed by a computer, cause the computer to carry out the computer-implemented method according to the invention for determining a solid fraction in a flowable, in particular aqueous, medium, in particular by means of a microwave sensor and/or the method according to the invention for generating a training data set for a machine learning model for determining a solid fraction δ of a solid in a flowable, in particular aqueous, medium, in particular for the machine learning model according to the invention.

The method according to the invention for configuring the microwave sensor according to the invention comprises the following method steps:
Receiving a parameter set at the microwave sensor; and
Replacing a parameter set, already provided, in particular at the factory, at the microwave sensor, of the machine learning model implemented in fixed form in the microwave sensor by the received parameter set, or
Implementing the received parameter set on the parameter-set-free machine learning model implemented in fixed form in the microwave sensor.

Determining the solid fraction in the medium using the machine learning model and the newly provided parameter set.

The invention is explained in greater detail with reference to the following figures, in which:

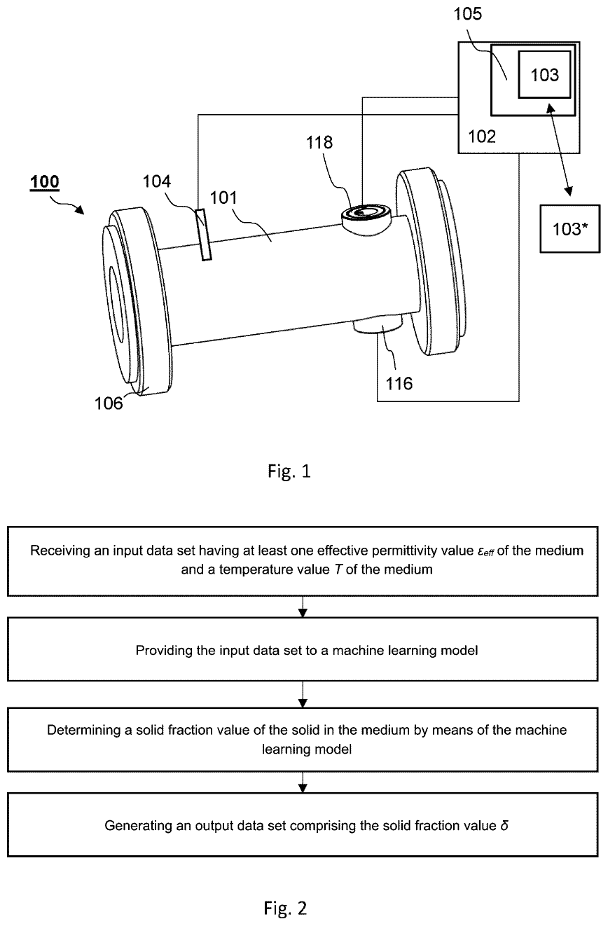

FIG. 1: shows a microwave sensor for determining a solid fraction δ of a solid in a flowable, in particular aqueous, medium;

FIG. 2: shows a process sequence of an embodiment of the method according to the invention for determining a solid fraction in a flowable, in particular aqueous, medium, in particular by means of a microwave sensor;

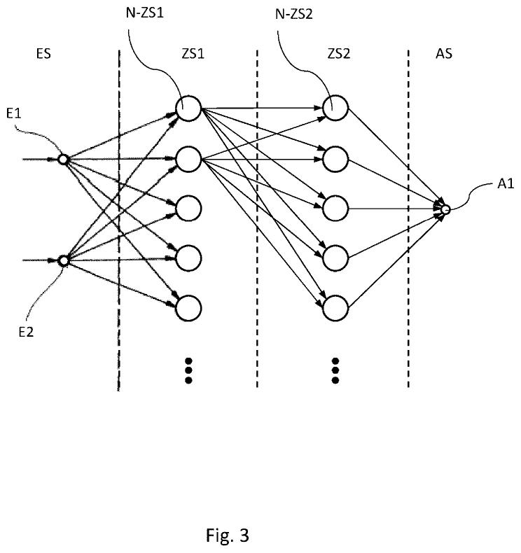

FIG. 3: shows a neural network of a method according to the invention according to a first embodiment in a schematic representation; and

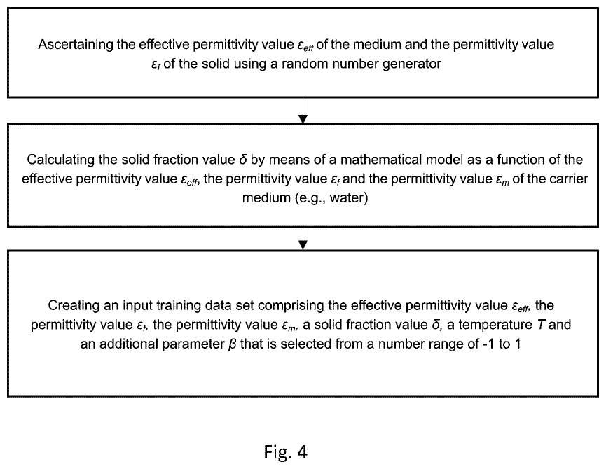

FIG. 4: shows a process sequence of an embodiment of the method according to the invention for generating a training data set for a machine learning model according to the invention for determining a solid fraction δ of a solid in a flowable, in particular aqueous, medium.

FIG. 1 shows a microwave sensor 100 for determining a solid fraction δ of a solid in a flowable, in particular aqueous, medium, having means for performing the method according to the invention for determining a solid fraction in a flowable, in particular aqueous, medium. In the application of the microwave sensor 100, the medium is guided through a measuring tube 101 in a process line.

The microwave sensor 100 shown itself comprises a transmitting antenna 116 for generating—if a medium is present in the measuring tube 101—a microwave signal that passes through the medium. The transmitting antenna 116 is arranged in an opening of the measuring tube 101. A suitable transmitting antenna 116 is disclosed in patent specification DE 10 2020 134 320 A1, to which reference is made in its entirety.

Furthermore, the microwave sensor 100 comprises a receiving antenna 118 for receiving the microwave signal generated by the transmitting antenna 116 and—if a medium is present in the measuring tube 101—passing through the medium. A suitable receiving antenna 118 is also disclosed in patent specification DE 10 2020 134 320 A1, to which reference is made in its entirety.

Alternatively, the microwave sensor 100 can comprise only one antenna, which is a receiving antenna 118 and simultaneously also a transmitting antenna 116. In this case, the microwave signal is generated by the antenna and also measured. For this purpose, the microwave signal is reflected at least once on an inner peripheral surface of the measuring tube back in the direction of the transmitting antenna.

The microwave sensor 100 also comprises a temperature sensor 104, which is arranged in an opening in the measuring tube 101 specially provided for this purpose. The temperature sensor is configured to ascertain a current temperature value of the guided medium and to provide it to an evaluation electronics unit 102. Alternatively, a temperature can be specified by the customer or the current temperature value can be ascertained by means of an external temperature sensor and provided to the evaluation electronics unit.

The evaluation electronics unit 102 is provided, which is configured to ascertain an effective permittivity ε_{eff} of the medium as a function of the provided propagation time and/or attenuation of the microwave signal. The attenuation results, for example, from the difference between a signal strength value of the received microwave signal and a signal strength value of the generated microwave signal. The receiving antenna 118 is configured to provide the received microwave signal to the evaluation electronics unit 102. The microwave signal generated and emitted by the transmitting antenna 116 is also provided to the evaluation electronics unit 102. The evaluation electronics unit 102 can be arranged in a microwave sensor housing (not shown), in which the transmitting antenna 116 and receiving antenna 118 are also arranged. An application-specific parameter set 103 is stored in the evaluation electronics unit 102, which is linked to a machine learning model. This is stored interchangeably, which means that the original or factory-stored parameter set 103 can be replaced by a parameter set 103* that differs therefrom. The evaluation electronics unit 102 is further configured to calculate the ascertained effective permittivity value ε_{eff} of the medium and a temperature value to a machine learning model 105 stored in the evaluation electronics unit 102. The machine learning model 105 comprises corresponding inputs for the provided effective permittivity value ε_{eff} and the provided temperature. Furthermore, the machine learning model 105 comprises an exchangeable, application-specific parameter set 103. This can, for example, be replaced by the customer with a parameter set 103* that differs from the parameter set 103 and is provided by the manufacturer. The parameter set 103 comprises at least one weighting factor wᵢ and at least one bias bᵢ. The machine learning model 105 is configured to calculate a solid fraction value δ of the solid in the medium and output it. The output can be effected via a display unit (with a display).

The evaluation electronics unit 102 is suitable for receiving a parameter set 103* and for replacing a parameter set 103 of the machine learning model 105 already provided, in particular at the factory, at the microwave sensor 100 and implemented in fixed form in the microwave sensor 100 with the received parameter set 103*. Alternatively, whenever no parameter set 103 has yet been provided, the received parameter set 103* can be implemented on the parameter-set-free machine learning model 105, which is implemented in fixed form in the microwave sensor 100, in particular in the evaluation electronics unit 102. The received parameter set 103* can be provided via a cloud or handheld device (tablet, smartphone, laptop) connected to the microwave sensor 100. Furthermore, the evaluation electronics unit 102 is configured to determine the solid fraction δ in the medium using the machine learning model 105 and the newly provided parameter set 103*.

FIG. 2 shows a process sequence of an embodiment of the computer-implemented method according to the invention for determining a solid fraction in a flowable, in particular aqueous, medium, in particular by means of a microwave sensor. The method comprises the following method step:
Receiving an input data set having at least one effective permittivity value ε_{eff}, in particular ascertained from measurement data, of the medium, and a temperature value T of the medium;

In addition, the input data set can further comprise a frequency value f for the microwave signal used and/or an effective density value ρ_{eff} of the medium.

The method further comprises the following method step:
Providing the input data set to a machine learning model.

Machine learning is a general term for the “artificial” creation of knowledge from experience. An artificial system (machine learning model) learns from provided examples and cases and can generalize them after the learning phase has ended. For this purpose, machine learning algorithms build a statistical model that is based on the input data sets and that is tested against the input data sets. Thus, the machine learning model recognizes patterns and regularities in the input data sets.

The machine learning model comprises an exchangeable, application-specific parameter set having at least one weighting factor wᵢ and at least one bias bᵢ. The input values entering the particular neuron are weighted with the corresponding weighting factor wᵢ (e.g., by multiplication). All weighted input values are then summed in the neuron. A bias bᵢ assigned to the neuron is then added to the sum. The result is then passed through an activation function in the subsequent step. The activation function can, for example, be a hyperbolic tangent function. Alternatively, the ReLU function can also be used. The output value resulting from this is then provided to all neurons of the next intermediate layer or to one neuron of the output layer. The output value; can be described by the following equation

$${\hat{y}}_{i} = {g}_{i} ( z ) = {g}_{i} ( {\sum }_{p = 1}^{P} {w}_{p} {x}_{p} + {b}_{i} )$$

gᵢ (z) denotes the activation function and P denotes the number of input values over which the summation is carried out.

The parameter set results from a mathematical model used in a learning phase of the machine learning model for describing the properties of heterogeneous mixtures. In the present case, these consist of an insoluble substance that is located in a carrier substance (e.g., water).

The mathematical model is selected from one of the listed models/equations:
the Maxwell-Garnett model
the parallel Wiener equation
the serial Wiener equation

The Maxwell-Garnett model describes the dielectric properties of bodies made of different materials having differing dielectric properties. The model assumes the following relationship

$$( \frac{{ε}_{eff} - {ε}_{m}}{{ε}_{eff} + 2 {ε}_{m}} ) = δ ( \frac{{ε}_{f} - {ε}_{m}}{{ε}_{f} + 2 {ε}_{m}} )$$

having a solid fraction value δ, an effective permittivity value ε_{eff} of the medium, the permittivity value ε_{f} of the solid and the permittivity value εₘ of the carrier medium (e.g., water).

The following relationship applies to the Wiener equation

$${ε}_{eff} = δ {ε}_{f}^{β} + ( 1 - δ ) {ε}_{m}^{β}$$

wherein the serial Wiener equation is based on β=−1, and the parallel Wiener equation is based on β=+1.

The method further comprises the following method step:
Determining a solid fraction value δ of the solid in the medium by means of the machine learning model.

The method further comprises the following method step:
Generating an output data set comprising the solid fraction value δ.

Optionally, the method can also comprise the following step:
Outputting the output data set, in particular the solid fraction value δ.

FIG. 3 shows a neural network of a method according to the invention according to a first embodiment in a schematic representation. A suitable machine learning model would be, for example, MLPRegressor. However, other machine learning models are also suitable for the method according to the invention. The machine learning model shown for determining a solid fraction in a flowable, in particular aqueous, medium, in particular for use in the computer-implemented method according to the invention, comprises an input layer ES having at least two inputs E1, E2. An effective permittivity value ε_{eff} of the medium is input into the machine learning model via input E1, and a temperature value T of the medium is input into the machine learning model via input E2. The machine learning model also comprises an output layer AS having an output A1 for a solid fraction value δ of the solid in the medium. Between the input layer and the output layer, there are at least two hidden intermediate layers, in each case having a number Nᵢ of connected neurons.

Alternatively (not shown), the input layer ES can comprise at least one further input for a frequency value f and/or a further input for an effective density value ρ_{ff}. The input or inputs are correspondingly connected to at least one of the neurons, in particular to all of the neurons, and provide, at the at least one neuron, in particular at all of the neurons, the corresponding density value ρ_{eff} and/or the frequency value.

In the embodiment shown, all neurons of an intermediate layer ZS1, ZS2 are exclusively networked with neurons of an adjacent intermediate layer. This means that there are no further interconnections to neurons in a non-adjacent intermediate layer. FIG. 3 shows 5 neurons (circle) per intermediate layer. However, the dotted lines indicate that more neurons can also be provided. The interconnection is represented by the arrows. For reasons of clarity, only the interconnection of the first two neurons with the neurons of the second intermediate layer ZS2 is shown. It is self-evident, however, that the other neurons are also interconnected.

The embodiment shown comprises exactly two intermediate layers. Alternatively, the machine learning model can also comprise more than two, in particular exactly three, intermediate layers. The machine learning model is selected so that the total number of neurons in the three intermediate layers is between 40 and 200 neurons. In order to make optimal use of the memory to be expended, it is advantageous if the number of neurons in the first intermediate layer is selected from the range of 20 to 80, the number of neurons in the second intermediate layer is selected from the range of 10 to 80 and the number of neurons in the third intermediate layer is selected from the range of 10 to 60.

In an alternative and equally advantageous embodiment, the machine learning model comprises exactly four intermediate layers having a total number of neurons of 100 to 250 neurons. In this case, the first intermediate layer comprises a number of neurons that is selected from the range of 20 to 60, the second intermediate layer comprises a number of neurons that is selected from the range of 50 to 80, the third intermediate layer comprises a number of neurons that is selected from the range of 20 to 60 and the fourth intermediate layer comprises a number of neurons that is selected from the range of 10 to 50.

The embodiments result in a machine learning model that comprises a data size of less than/equal to 500 KB, with a deviation of less than 0.1% relative to the mathematical model serving as the basis for the learning phase. If more than 500 KB of memory is available, up to 1500 neurons can be provided with two fixed inputs (effective permittivity and temperature).

The machine learning model shown is configured to weight input values received in each neuron of a first intermediate layer of the at least two intermediate layers in each case with a weighting factor, to sum the weighted input values and a bias bᵢ to a sum value, to convert the sum value into an output value through an activation function and to provide the output value to at least one neuron, in particular to all neurons, a second intermediate layer of the at least two intermediate layers or to the output layer AS. The weighting factors wᵢ and the bias bᵢ are part of the exchangeable, application-specific parameter set. The activation function can be a hyperbolic tangent function and/or a ReLU (rectified linear unit) function. Alternatively, the activation function can also be an exponential linear unit (ELU) or a sigmoid function.

FIG. 4 shows a process sequence of an embodiment of the method according to the invention for generating a training data set for a machine learning model according to the invention for determining a solid fraction δ of a solid in a flowable, in particular aqueous, medium. The method comprises the method step:
Ascertaining at least the effective permittivity value ε_{eff} of the medium and the permittivity value ε_{f} of the solid using a random number generator.

The random number generator is subject to a probability distribution, such as the normal distribution. The randomly generated permittivity values result in a data set that flows into the next method step:
Calculating the solid fraction value δ by means of a mathematical model as a function of the effective permittivity value ε_{eff}, the permittivity value ε_{f} and the permittivity value εₘ of the carrier medium.

The mathematical model is selected from one of the listed models:
the Maxwell-Garnett model
the parallel Wiener equation
the serial Wiener equation.

For example, the Maxwell-Garnett model can be used for the training data set. For this purpose, the Maxwell-Garnett equation is rearranged so that the effective permittivity can be calculated as a function of the remaining quantities. Furthermore, the permittivity of the solid can be fixed. A value between 2 and 5 can be assumed for the permittivity of the solid. The permittivity of the carrier medium (e.g., water) is temperature dependent. Thus, the temperature is included in the calculation of the permittivity of the carrier medium (e.g., through the Debye model). For the solid fraction, a value from a range that is reasonable for the application (e.g., 0-35% TS) is assumed.

The next method step comprises:
Creating an input training data set comprising an effective permittivity value ε_{eff}, a permittivity value ε_{f}, a permittivity value εₘ, a solid fraction value δ, a temperature T and an additional parameter β that is selected from a number range of −1 to 1.

For this method step, the Wiener equation is used as a model for describing the medium, and the additional parameter β is therefore employed. Alternatively, the Maxwell-Garnett model can also be used.

Alternatively, the input training data set can further comprise an effective density p of the medium. The temperatures are advantageously selected from a temperature range of 1 to 100° C., in particular 10 to 60° C. The effective permittivity value ε_{eff} and the permittivity value ε_{f} are selected so that a solid fraction value δ between 0 and 50% (inclusive) results—as is to be expected, for example, in wastewater applications. However, the limit can also be higher. Applications are known in which a solid fraction of more than 50% is possible. In that case, the input training data set would comprise solid fraction values δ between 0 and the upper limit relevant for the application.

1-21. (canceled)

22. A computer-implemented method for determining a solid fraction in a flowable, aqueous, medium, using a microwave sensor, comprising:
receiving an input data set having at least one effective permittivity value ε_{eff}, ascertained from measurement data, of the medium, and a temperature value T of the medium;
providing the input data set to a machine learning model,
wherein the machine learning model comprises an exchangeable, application-specific parameter set,
wherein the parameter set comprises at least one weighting factor wᵢ and at least one bias bᵢ;
determining a solid fraction value δ of the solid in the medium using the machine learning model;
generating an output data set comprising the solid fraction value δ;
outputting the solid fraction value δ.

23. The method according to claim 22,
wherein the input data set further comprises a frequency value f and/or an effective density value ρ_{eff} of the medium.

24. The method according to claim 23,
wherein the parameter set results from a mathematical model applied in a learning phase of the machine learning model for describing the properties of heterogeneous mixtures consisting of an insoluble substance located in a carrier substance.

25. The method according to claim 24,
wherein the mathematical model is selected from:
the Maxwell-Garnett model
the parallel Wiener equation
the serial Wiener equation.

26. A machine learning model for determining a solid fraction in a flowable, in particular aqueous, medium, in particular for use in the method according to claim 22, comprising:
an input layer having at least two inputs for an effective permittivity value ε of the medium, and a temperature value T of the medium;
an output layer having an output for a solid fraction value δ of the solid in the medium;
at least two hidden intermediate layers present between the input layer and the output layer, in each case having a number Nᵢ of connected neurons,
wherein the machine learning model is configured to weight input values received in each neuron of a first intermediate layer of the at least two intermediate layers in each case with a weighting factor, to sum the weighted input values and a bias b; to form a sum value, to convert the sum value into an output value through an activation function and to provide the output value to at least one neuron, in particular to all neurons, of a second intermediate layer of the at least two intermediate layers,
wherein the weighting factors wᵢ and the bias bᵢ form an exchangeable, application-specific parameter set.

27. The machine learning model according to claim 26,
wherein a hyperbolic tangent function and/or a ReLU (rectified linear unit) function is used as the activation function.

28. The machine learning model according to claim 26,
wherein the input layer comprises at least one further input for a frequency value f and/or an effective density value ρ_{eff}.

29. The machine learning model according to one of claim 26,
wherein the machine learning model comprises exactly three intermediate layers,
wherein a total number of neurons of the three intermediate layers is selected from a range of 40 to 2000, in particular up to 1000, and preferably up to 200 neurons.

30. The machine learning model according to claim 29,
wherein a first intermediate layer comprises a number of neurons selected from the range of 20 to 80,
wherein a second intermediate layer comprises a number of neurons selected from the range of 10 to 80,
wherein a third intermediate layer comprises a number of neurons selected from the range of 10 to 60.

31. The machine learning model according to claim 26,
wherein the machine learning model comprises exactly four intermediate layers,
wherein a total number of the neurons of the four intermediate layers is selected from a range of 100 to 250 neurons.

32. The machine learning model according to claim 31,
wherein a first intermediate layer comprises a number of neurons selected from the range of 20 to 60,
wherein a second intermediate layer comprises a number of neurons selected from the range of 50 to 80,
wherein a third intermediate layer comprises a number of neurons selected from the range of 20 to 60,
wherein a fourth intermediate layer comprises a number of neurons selected from the range of 10 to 50.

33. The machine learning model according to claim 26,
wherein the machine learning model comprises a data size of less than or equal to 500 kB, with a deviation of less than 0.1% relative to the mathematical model serving as the basis for the learning phase.

34. A computer-implemented method for generating a training data set for a machine learning model for determining a solid fraction δ of a solid in a flowable, aqueous, medium, for the machine learning model according to one of claims 5 to 12, comprising:
creating an input training data set,
wherein the input training data set comprises an effective permittivity value ε_{eff} of the medium, a permittivity value ε_{f} of the solid, a permittivity value εₘ of the carrier medium, a solid fraction value δ and a temperature T,
wherein at least the effective permittivity value ε_{eff} and the permittivity value ε_{f} are ascertained by a random number generator,
wherein the solid fraction value δ is calculated by means of a mathematical model as a function of the effective permittivity value ε_{eff}, the permittivity value ε_{f} and the permittivity value εₘ.

35. The computer-implemented method according to 34,
wherein the input training data set further comprises an additional parameter β, which is selected from a number range of −1 to 1.

36. The computer-implemented method to claim 34,
wherein the mathematical model is selected from:
the Maxwell-Garnett model
the parallel Wiener equation
the serial Wiener equation.

37. The computer-implemented method according to claim 34,
wherein the input training data set further comprises an effective density ρ_{eff} of the medium.

38. The computer-implemented method according to claim 34,
wherein the input training data set is created for temperatures from a temperature range of 1 to 100° C., in particular 10 to 60° C., and/or
wherein the effective permittivity value ε_{eff} and the permittivity value ε_{f} are selected so that a solid fraction value δ between 0 and 35% results.

39. A microwave sensor for determining a solid fraction δ of a solid in a flowable, in a particular aqueous, medium, having means for performing the method according claim 22, comprising:
a transmitting antenna for generating a microwave signal passing through the medium;
a receiving antenna for receiving the microwave signal passing through the medium,
an evaluation electronics unit that is configured to ascertain an effective permittivity of the medium as a function of a provided propagation time and/or attenuation of the microwave signal,
wherein the application-specific parameter set is stored interchangeably in the evaluation electronics unit.

40. A computer program comprising instructions that, when executed by a computer, cause the computer to carry out the method according to one of claim 22.

41. A computer-readable medium containing instructions that, when executed by a computer, cause the computer to carry out the method of one of claim 22.

42. A method for configuring a microwave sensor according to claim 18, comprising the following method steps:
receiving a parameter set at the microwave sensor; and
replacing a parameter set, already provided, at the microwave sensor, of the machine learning model implemented in fixed form in the microwave sensor by the received parameter set, or
implementing the received parameter set on the parameter set-free machine learning model implemented in fixed form in the microwave sensor.
determining the solid fraction in the medium using the machine learning model and the newly provided parameter set.
